(19) United States
(12) Patent Application Publication
(10) Pub. No.: US 2024/0194310 A1
(43) Pub. Date: Jun. 13, 2024

(54) VACCINE MANAGEMENT DEVICE AND VACCINE MANAGEMENT SYSTEM INCLUDING THE SAME, OPERATING METHOD OF VACCINE MANAGEMENT DEVICE AND METHOD FOR PROVIDING VACCINE HEALTHCARE SOLUTIONS

(71) Applicant: Real Time Medi Check Corp., Wonju-si, Gangwon-do (KR)

(72) Inventors: Hee KIM, Incheon (KR); Jae Joon LIM, Incheon (KR)

(21) Appl. No.: 18/554,245

(22) PCT Filed: Apr. 6, 2022
(86) PCT No.: PCT/KR2022/004912

§ 371 Date: Oct. 18, 2023

(30) Foreign Application Priority Data
Apr. 7, 2021 (KR) 10-2021-0045323
Apr. 7, 2021 (KR) 10-2021-0045324
Apr. 7, 2021 (KR) 10-2021-0045325

(51) Int. Cl. G16H 10/60; G16H 10/20; G16H 20/17

(52) U.S. Cl. CPC G16H 10/60; G16H 10/20; G16H 20/17

(57) ABSTRACT

An operating method of a vaccine management device of the present invention includes: waiting, by the identification code recognition unit, recognition of an identification code; determining, when a first identification code is recognized in the first waiting step, whether the first identification code is a subject person identification code of a vaccine inoculation subject person; waiting, when the first identification code is the subject person identification code, recognition of a vaccine identification code; determining, when a second identification code is recognized in the second waiting step, whether the second identification code is the vaccine identification code; and transmitting, when the second identification code is the vaccine identification code, data obtained by merging subject person identification information corresponding to the subject person identification code and vaccine identification information corresponding to the vaccine identification code, to a management server.

### CROSS-REFERENCE TO RELATED APPLICATIONS

This application is a National Stage Entry of International Application No. PCT/KR2022/004912 filed Apr. 6, 2022, which claims priority from Korean Application Nos. 10-2021-0045323 filed Apr. 7, 2021, 10-2021-0045324 filed Apr. 7, 2021, and 10-2021-0045325 filed Apr. 7, 2021. The aforementioned applications are incorporated herein by reference in their entireties.

### TECHNICAL FIELD

The embodiments of the present invention relate to a vaccine management system including a vaccine management device, which can easily manage a inoculation subject person of a vaccine (or a person who inoculated with the vaccine) and a history of the vaccine by unifying the inoculation subject person of the vaccine and history management of the vaccine, an operating method of the vaccine management device, and a vaccine healthcare solution providing method performed by the vaccine management system.

### BACKGROUND ART

A vaccine has characteristics that have stricter distribution and storage conditions and a shorter shelf life compared to other logistics. In particular, the vaccine for specific infectious diseases (example, Corona 19) should be stored and distributed in an ultra low-temperature environment such as minus 20 degrees or less, and multiple inoculations should be carried out, not a single inoculation.

Meanwhile, multiple inoculations are carried out at an interval of a specific period, and in this case, vaccines are feared to be inoculated differently. In particular, in the case of vaccines which are approved for emergency use without a sufficient verification procedure to prevent the spread of new infectious diseases, an inoculation subject person avoids vaccine inoculation due to anxiety about the side effects of vaccine inoculation, the limitations of cause estimation of the side effects, and a responsibility avoidance policy of a vaccine manufacturer on the side effects. Moreover, subject persons are excluded from the flow of information related to the vaccine, and some inoculation subject persons are being damaged by vaccine information management errors.

Therefore, technology is desperately required, which can make the overall vaccine inoculation management simpler and clearer, provide a vaccine healthcare service to the inoculation subject person, and secure scientific basis data for pre and post vaccine managements when side effects of vaccine inoculation occur.

### DISCLOSURE

### Technical Problem

An object of the present invention is to provide a vaccine management system including a vaccine management device, which can establish a stable vaccine prescription system by identifying and managing the inoculation history of vaccine syringes for each use location together with inoculation subject person identification information in real time, an operating method of the vaccine management device, and a vaccine healthcare solution providing method performed by the vaccine management system.

The technical problems of the present invention are not limited to the technical problems described above, and other technical problems not described herein will be clearly understood by those skilled in the art from the description below.

### Technical Solution

A method according to an embodiment of the present invention is an operating method of a vaccine management device including an identification code recognition unit, the method including: a first waiting step of waiting, by the identification code recognition unit, recognition of an identification code; a first determination step of determining, when a first identification code is recognized in the first waiting step, whether the first identification code is a subject person identification code of a vaccine inoculation subject person; a second waiting step of waiting, when the first identification code is the subject person identification code, recognition of a vaccine identification code; a second determination step of determining, when a second identification code is recognized in the second waiting step, whether the second identification code is the vaccine identification code; and a merged information transmission step of transmitting, when the second identification code is the vaccine identification code, data obtained by merging subject person identification information corresponding to the subject person identification code and vaccine identification information corresponding to the vaccine identification code, to a management server.

A system according to an embodiment of the present invention is a vaccine management system, the system including: a management server, at least one vaccine management device, and at least one inoculation subject person terminal, wherein the inoculation subject person terminal collects, from an inoculation subject person, basic information of the inoculation subject person; the management server generates an inoculation subject person identification code based on the basic information of the inoculation subject person, and transmits the inoculation subject person identification code to the inoculation subject person terminal; the vaccine management device sequentially scans the inoculation subject person identification code output to the inoculation subject person terminal and a vaccine identification code provided in a vaccine, and transmits, to the management server, merged data including inoculation subject person information de-identification data acquired from the inoculation subject person identification code and vaccine information data acquired from the vaccine identification code; and the management server generates a vaccine inoculation history of the inoculation subject person based on the inoculation subject person information de-identification data and the vaccine information data, and transmits, to the inoculation subject person terminal, a vaccine inoculation history message including the vaccine inoculation history.

A vaccine healthcare solution providing method according to another embodiment of the present invention includes the steps of: collecting, by an inoculation subject person terminal, from an inoculation subject person, basic information of the inoculation subject person; generating, by a management server, an inoculation subject person identification code based on the basic information of the inoculation subject person; transmitting, by the management server, the inoculation subject person identification code to the inoculation subject person terminal; sequentially scanning, by a vaccine management device, the inoculation subject person identification code output to the inoculation subject person terminal and a vaccine identification code provided in a vaccine; transmitting, by the vaccine management device, to the management server, merged data including inoculation subject person information de-identification data acquired from the inoculation subject person identification code and vaccine information data acquired from the vaccine identification code; generating, by the management server, a vaccine inoculation history of the inoculation subject person based on the inoculation subject person information de-identification data and the vaccine information data; and transmitting, by the management server, to the inoculation subject person terminal, a vaccine inoculation history message including the vaccine inoculation history.

### Advantageous Effects

According to the present invention, it may efficiently manage the vaccine history for each inoculation subject person in a more improved environment.

In addition, according to the present invention, it may simplify an operating algorithm of the vaccine management device for the vaccine history management for each inoculation subject person, and may minimize the number and the amount of data transmitted and received between the vaccine management device and the management server, thereby performing optical data management.

In addition, according to the present invention, it may recheck the suitability of the vaccine to be inoculated through the vaccine management device, so that incompatible vaccine inoculations such as interchangeable use of multiple inoculation vaccines or inoculation of vaccines with poor cold chain storage and distribution history are fundamentally blocked, thereby establishing a stable prescription system for vaccines.

In addition, according to the present invention, it may identify and manage the exhaustion of vaccine syringes for use location in real time to establish a stable and reasonable supply system for vaccines, thereby preventing ongoing vaccine shortage phenomenon or over-inventory occurrence phenomenon, and efficiently performing inventory management and demand prediction of vaccines.

### BRIEF DESCRIPTION OF THE DRAWINGS

FIG. 1 is a diagram schematically illustrating a structure of a vaccine management system according to an embodiment of the present invention.

FIG. 2 is a flowchart exemplarily illustrating a method for providing a vaccine healthcare solution according to an embodiment of the present invention.

FIG. 3 is a flowchart exemplarily illustrating a process of collecting basic information of a subject person by the vaccine healthcare solution according to an embodiment of the present invention.

FIG. 4 is a diagram exemplarily illustrating a method for generating a subject person identification code used in the vaccine management system according to an embodiment of the present invention.

FIG. 5 is a diagram for conceptually describing the use state of a vaccine management device according to an embodiment of the present invention.

FIGS. 6 to 8 are flowcharts for describing an operating method of the vaccine management device according to the embodiments of the present invention.

FIG. 9 is a diagram for exemplarily describing a service provided by a management server according to an embodiment of the present invention.

FIG. 10 is a diagram exemplarily illustrating a management method of an inoculation subject person identification code used in the vaccine management system according to an embodiment of the present invention.

### BEST MODE

Hereinafter, preferred embodiments of the present invention will be described in detail with reference to the accompanying drawings. Advantages and features of the present invention, and methods for accomplishing the same will become apparent with reference to the embodiments to be described below in detail together with the accompanying drawings. However, the present invention is not limited to the embodiments disclosed below, but may be embodied in various different forms. The present embodiments are provided only to make the disclosure of the present invention complete and fully inform those skilled in the art of the scope of the invention, and the present invention is merely defined by the scope of the claims. Throughout the whole specification, the same reference numerals denote the same elements.

Unless otherwise defined, all terms (including technical and scientific terms) used herein may be used as the meaning which may be commonly understood by those skilled in the art. In addition, the terms defined in commonly used dictionaries will not be construed in an idealized or excessive sense unless expressly and specifically defined.

In addition, singular forms used herein may also include plural forms unless specifically stated in the phrase. The terms “comprises” and/or “comprising” used herein are intended to designate the presence of stated components, steps, operations and/or elements, but not to preclude the possibility of the presence or addition of one or more other components, steps, operations and/or elements.

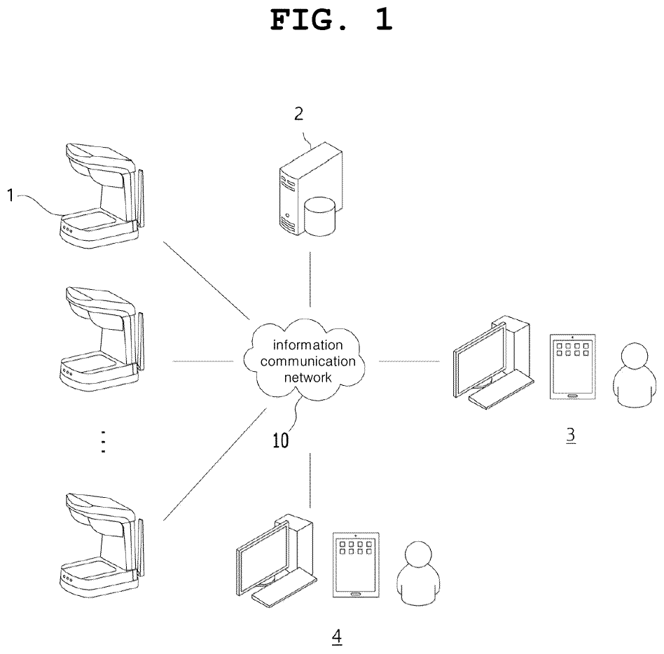

FIG. 1 is a diagram schematically illustrating a structure of a vaccine management system according to an embodiment of the present invention.

Referring to FIG. 1, the vaccine management system according to an embodiment of the present invention may include a plurality of vaccine management devices 1 connected to a management server 2 through an information communication network 10 and providing vaccine information used in real time and information on an inoculation subject person (hereinafter, referred to as “subject person”) of vaccine, and a medical staff interface 3 and a subject person interface 4 connected to the management sever 2 and/or a manager interface through the information communication network 10.

Although not shown, the vaccine management system may further include a manager interface that monitors and manages a vaccine use status in each of the plurality of vaccine management devices 1 collected in the management server 2. The manager interface may be implemented as a separate entity from the management server 2 or as a single device that is physically same as the management server 2.

Furthermore, although not shown, the vaccine management system may be connected to a vaccine informatization system (e.g., a health insurance management system, drug utilization review (DUR), etc.) through the information communication network 10, and may also collect, process, and use vaccine prescription history information for each medical institution.

Each of the manager interface, the medical staff interface 3, and the subject person interface 4 may be a wireless communication device that ensures portability and mobility. Alternatively, each of the manager interface, the medical staff interface 3, and the subject person interface 4 may be implemented by a desktop, a notebook, a laptop, etc., equipped with a web browser (WEB Browser). Alternatively, each of the manager interface, the medical staff interface 3, and the subject person interface 4 may be implemented as various smart devices such as smartphones, smart notes, tablet PCs, smart cameras, smart TVs, and wearable computers.

The information communication network 10 may be implemented, for example, as a wired network such as a local area network (LAN), a wide area network (WAN) or a value added network (VAN), or all types of wireless networks such as a mobile radio communication network, a satellite communication network, Bluetooth, wireless broadband Internet (Wibro), high speed downlink packet access (HSDPA), etc.

The management server 2 may be a server device, including a high-performance processor for storing and processing vaccine information and/or subject person information input into each of the plurality of vaccine management devices 1, and for processing a response procedure without delay in response to the request of the vaccine management devices 1 if necessary, and a workstation having a large capacity storage space.

The management server 2 may perform communication with at least one of the vaccine management devices 1, the medical staff interface 3, and the subject person interface 4, and collect and manage vaccine inoculation-related information.

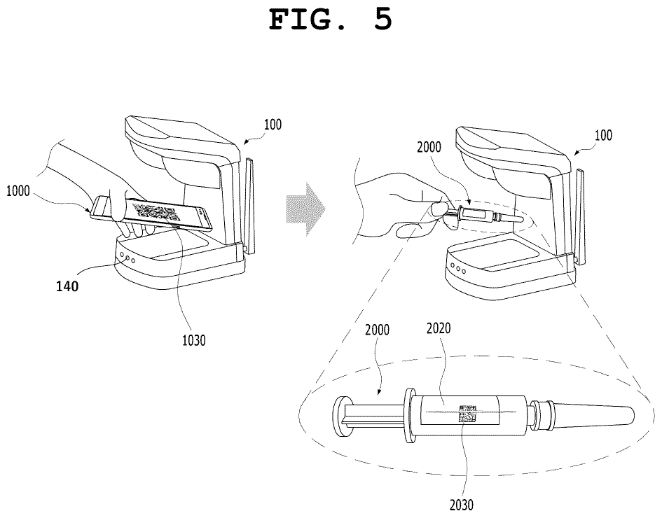

Specifically, the management server 2 may collect basic information of the subject person, and generate a unique identification code for each subject person based on the collected basic information of the subject person. Further, the management server 2 may collect and manage a subject person identification code 1030 (see FIG. 5) and a vaccine identification code 2030 (see FIG. 5) recognized by each vaccine management device 1.

Furthermore, the management server 2 may generate and analyze big data based on vaccine information cumulatively used for each vaccine management device 1. As a result, an optimal vaccine demand amount for period/use location may be predicted, and also, vaccine inventory management may be automated and processed based on the predicted demand amount.

Through the medical staff interface 3, a medical staff may manage a subject person identification code generated or updated for each subject person. For example, the medical staff may manage the subject person in medical sites through the subject person identification code 1030 (see FIG. 5) assigned for each subject person. The medical staff interface 3 may be implemented in a medical staff terminal.

In addition, the medical staff interface 3 may control a printing device capable of printing the subject person identification code 1030 in medical sites. Further, by using the medical staff interface 3, the medical staff may monitor the status of vaccines managed by the vaccine management device 1 or perform inventory management such as vaccine orders.

The subject person interface 4 may receive identity information for issuing the subject person identification code 1030, output the issued subject person identification code 1030 a subject person terminal display, or perform post management of the subject person. The post management of the subject person may include providing history information of vaccine inoculation, collecting self-diagnosis details after inoculation, and providing notification information of secondary inoculation information. The subject person interface 4 may be implemented in the medical staff terminal.

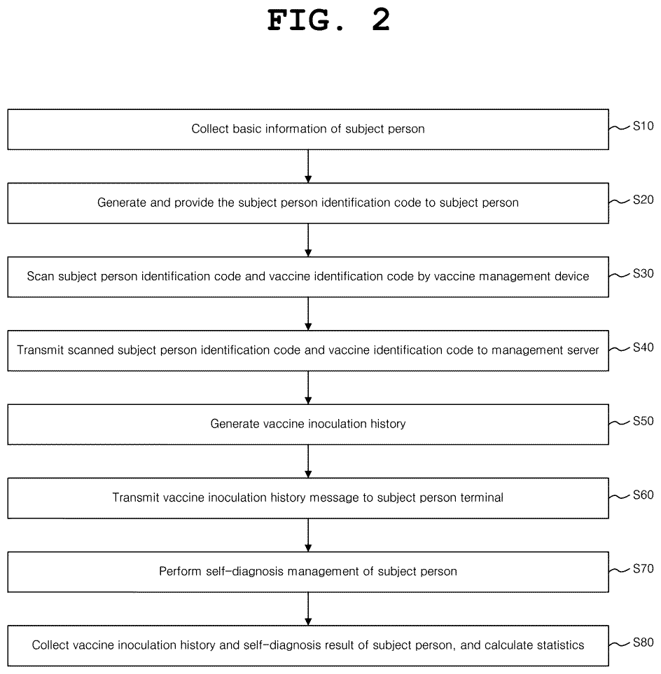

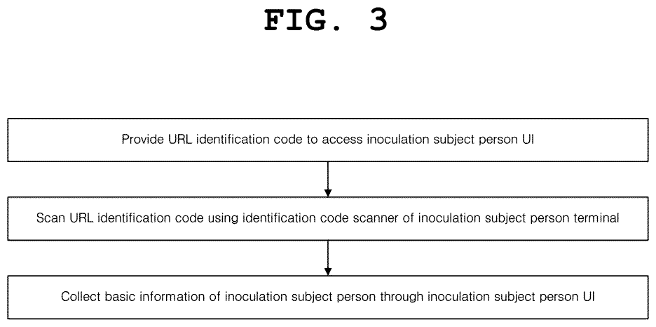

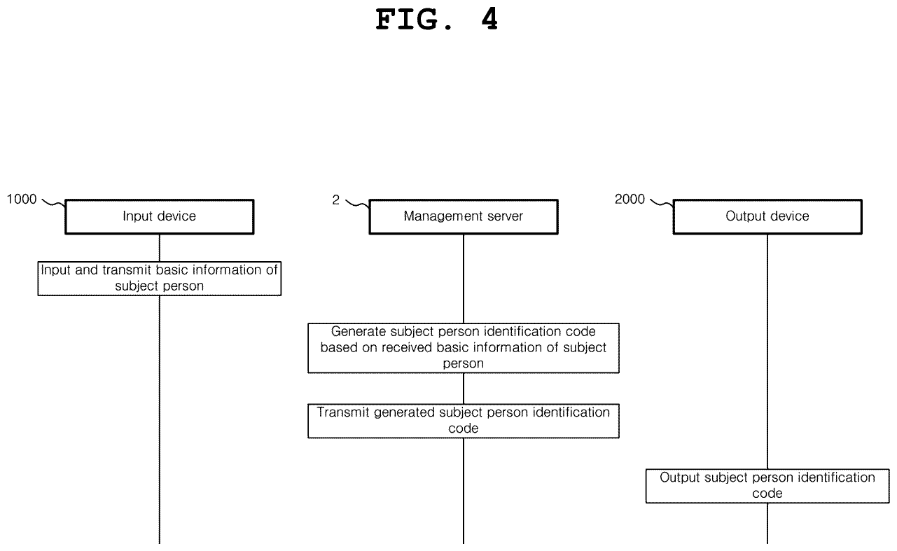

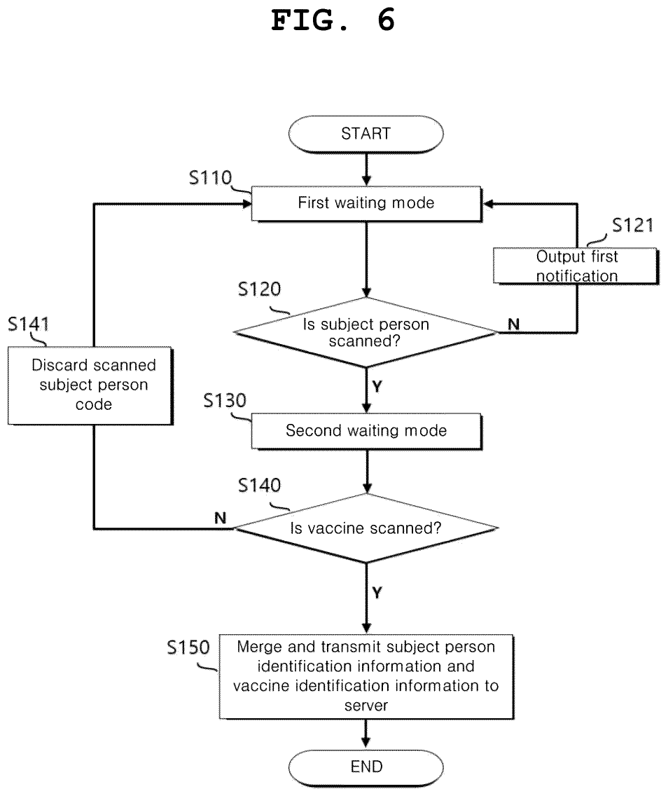

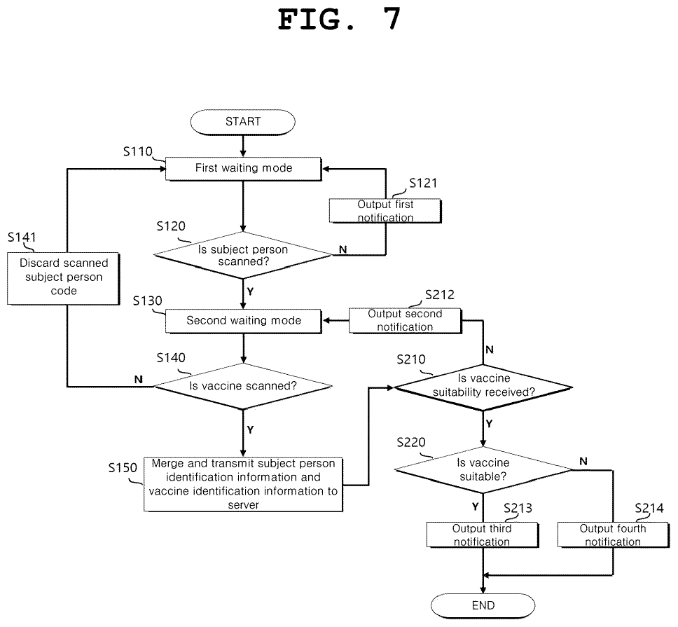

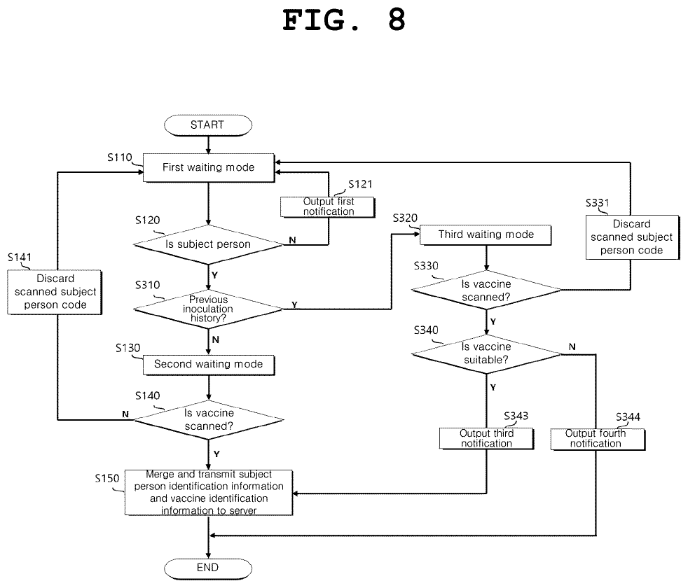

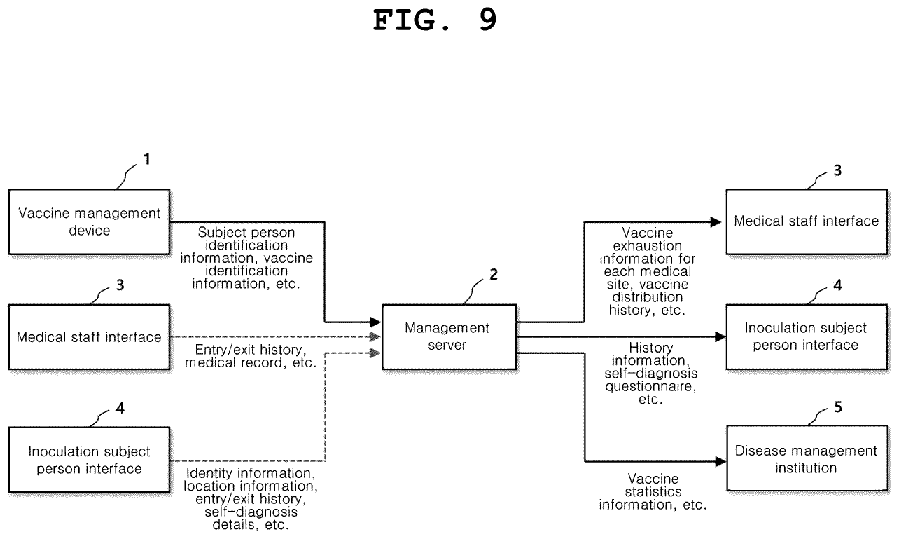

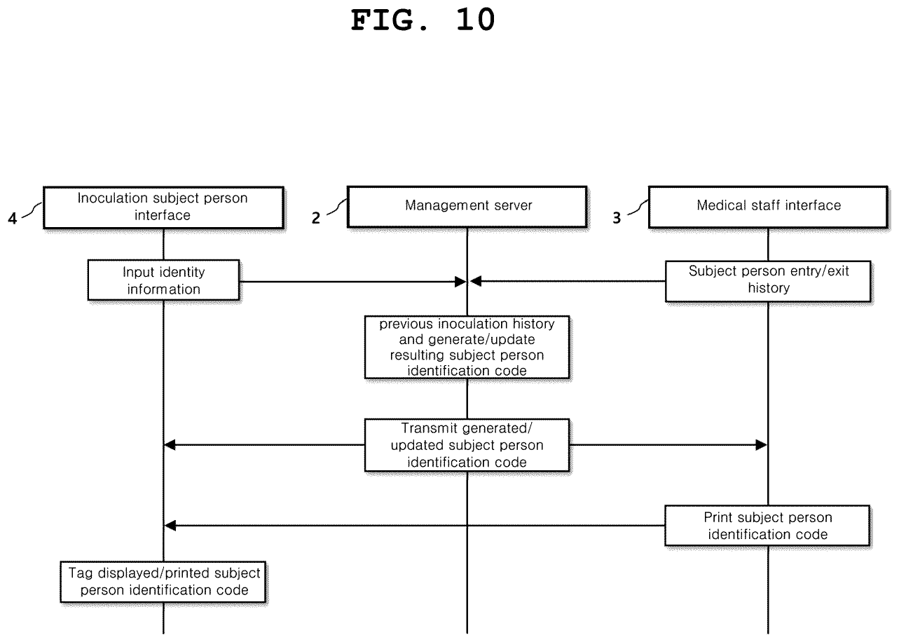

FIG. 2 is a flowchart exemplarily illustrating a method for providing a vaccine healthcare solution according to an embodiment of the present invention, FIG. 3 is a flowchart exemplarily illustrating a process of collecting basic information of a subject person by the vaccine healthcare solution according to an embodiment of the present invention, FIG. 4 is a diagram exemplarily illustrating a method for generating a subject person identification code used in the vaccine management system according to an embodiment of the present invention, FIG. 5 is a diagram for conceptually describing the use state of a vaccine management device 1 according to an embodiment of the present invention, FIG. 6 is a flowchart for describing an operating method of the vaccine management device 1 according to an embodiment of the present invention, FIG. 7 is a flowchart for describing an operating method of the vaccine management device 1 according to another embodiment of the present invention, FIG. 8 is a flowchart for describing an operating method of the vaccine management device 1 according to still another embodiment of the present invention, FIG. 9 is a diagram for exemplarily describing a service provided by a management server 2 according to an embodiment of the present invention, and FIG. 10 is a diagram exemplarily illustrating a management method of a subject person identification code used in the vaccine management system according to an embodiment of the present invention.

Referring to the drawings, in particular, FIG. 2, the method for providing the vaccine health care solution may include a step S10 of collecting basic information of the subject person, a step S20 of generating and providing a subject person identification code to the subject person, a step S30 of receiving a subject person identification code and a vaccine identification code scanned by a vaccine management device, a step S40 of transmitting scanned inoculated person identification information and vaccine identification information to a management server, a step S50 of generating a vaccine inoculation history based on a subject person information de-identification data and a vaccine information data, a step S60 of transmitting a vaccine inoculation history message to an inoculated person terminal, a step S70 of managing self-diagnosis of the inoculated person, and a step S80 of collecting and statistically calculating a vaccine inoculation history and a self-diagnosis result of the inoculated person.

In step S10 of collecting the basic information of the subject person, the management server 2 may variously collect the basic information of the subject person.

According to the embodiment, the basic information of the subject person may be directly collected from the subject person. In this case, an input device 1000 of FIG. 4 may be the subject person terminal.

Specifically, referring to FIG. 3, a uniform resource locator (URL) identification code for issuing the subject person identification code required for the subject person interface 4 to access the management server 2 may be provided online and/or offline to medical sites, and the subject person may scan the URL identification code by using an identification code scanner (e.g., a smartphone camera) provided on the subject person terminal. Accordingly, the subject person terminal may access the management server 2, and a basic information collection interface corresponding to the URL identification code may be displayed on the subject person terminal. The basic information collection interface is included in the subject person interface 4. The subject person may input his/her basic information into the basic information collection interface, and accordingly, the management server 2 may collect the basic information of the subject person.

Alternatively, the subject person may input his/her basic information by directly accessing the vaccine management system by using the subject person terminal, and accordingly, the management server 2 may collect the basic information of the subject person. Alternatively, a vaccine healthcare application may be installed in the subject person terminal, and the vaccine healthcare application may be executed in link with the management server 2, and the management server 2 may also collect the basic information of the subject person by using the vaccine healthcare application.

According to another embodiment, the basic information of the subject person may also be indirectly collected from the subject person. For example, the medical staff may verbally request the basic information of the subject person from the subject person, and the medical staff may input the basic information of the subject person into the medical staff interface 3, and accordingly, the management server 2 may also collect the basic information of the subject person. In this case, the input device 1000 of FIG. 4 may be the medical staff terminal.

Meanwhile, the basic information of the subject person may be real name information including personal information (e.g., name, gender, and contact point, etc.) of the subject person, or false name information not including the personal information of the subject person.

Referring back to FIG. 2, in step S20 of generating and providing the subject person identification code to the subject person, the management server 2 may generate the subject person identification code 1030 based on the collected basic information of the subject person.

When generating the subject person identification code 1030, the management server 2 may de-identify the basic information of the subject person. Specifically, the management server 2 generates subject person information de-identification data based on the basic information of the subject person, thereby providing the subject person identification code 1030 having a format such as a bar code or a quick response (QR) code to the subject person. That is, the management server 2 may exclude personal information of the subject person included in identity information of the subject person, encrypt the identity information, and transmit the encrypted identity information to an output device 2000, and the subject person information de-identification data may be re-identified in the management server 2, the medical staff interface 3, or the vaccine informatization system (e.g., health insurance management system, DUR, etc.) if necessary in the future.

Meanwhile, the subject person identification code 1030 may be provided to the subject person in various ways. As an example, the subject person identification code 1030 may be output from the subject person interface 4 executed in the subject person terminal. However, the subject person identification code 1030 is not limited thereto, and the subject person identification code 1030 may also be output in the form of a paper printout by a printing device provided in medical sites. That is, the output device 2000 of FIG. 4 may be the subject person terminal display, in which the subject person interface 4 is implemented, and may also be a printing device such as an identification code printer provided in medical sites.

Hereinafter, step S20 will be described in detail with reference to FIG. 4.

FIG. 4 is a diagram exemplarily illustrating a method for generating a subject person identification code 1030 used in the vaccine management system according to an embodiment of the present invention.

Referring to FIG. 4, the input device 1000 may directly/indirectly receive the identity information input from the subject person, and transmit the input identity information to the management server 2. According to the embodiment, the identity information may include a name, a gender, and a contact point of the subject person. Here, the input device 1000 may be the subject person terminal or the medical staff terminal. When the input device 1000 is the subject person terminal, the subject person interface 4 may directly receive the identity information input from the subject person, and when the input device 1000 is the medical staff terminal, the subject person interface 4 may also indirectly receive the identity information confirmed from the subject person through the medical staff interface 3.

The management server 2 may convert the received subject person identity information into the subject person identification code 1030, and transmit the converted subject person identification code 1030 to the output device 2000. Here, the management server 20 may encrypt or de-identify, and convert the received identity information into the subject person identification code 1030. That is, the management server 2 may exclude and transmit personal information of the subject person included in the identity information of the subject person to the output device 2000, and the de-identified subject person identification code may also be re-identified in the medical staff interface 3 and/or the vaccine informatization system (e.g., health insurance management system, DUR, etc.) if necessary in the future.

The output device 2000 may output the received subject person identification code 1030, and the output subject person identification code 1030 may be thereafter identified through the vaccine management device 1. According to the embodiment, the output device 2000 may be the printing device such as the identification code printer provided in medical sites or the subject person terminal display with the subject person interface 4. That is, the subject person identification code 1030 may be output in printout in medical sites, or may be output by the subject person terminal display.

Referring back to FIG. 2, in step S30 of scanning the subject person identification code and the vaccine identification code by the vaccine management device, the vaccine management device 1 may sequentially scan the subject person identification code 1030 and the vaccine identification code 2030. In step S40 of transmitting the scanned subject person identification information and vaccine identification information to the management server, the vaccine management device 1 may generate merged data based on subject person identification information obtained from the scanned subject person identification code 1030 (i.e., an identification code of an inoculated person) and vaccine identification information obtained from the vaccine identification code 2030, and transmit the merged data to the management server 2.

Step S30 and step S40 may correspond to the operating method of the vaccine management device 1 shown in FIGS. 6 to 8.

Referring to FIGS. 5 and 6, the operating method of the vaccine management device 1 according to an embodiment of the present invention may include a first waiting step S110 of waiting, by an identification code recognition unit provided in the vaccine management device 1, recognition of an identification code, a first determination step S120 of determining, when a first identification code is recognized in the first waiting step, whether the first identification code is a subject person identification code 1030, a second waiting step S130 of waiting, when the first identification code is the subject person identification code 1030, recognition of a vaccine identification code 2030, a second determination step S140 of determining, when a second identification code is recognized in the second waiting step, whether the second identification code is the vaccine identification code 2030, and a merged information transmission step S510 of transmitting, when the second identification code is the vaccine identification code 2030, data obtained by merging the subject person identification information corresponding to the subject person identification code 1030 and the vaccine identification information corresponding to the vaccine identification code 2030, to the management server 2. Here, the first and the second identification codes may be the bar code or the QR code, but are not limited thereto.

The first waiting step S110 (or a first waiting mode) may be a step in which the identification code recognition unit of the vaccine management device 1 waits recognition of the subject person identification code 1030. The subject person identification code 1030 recognized in the first waiting step S110 may be an identification code output to the subject person terminal display or an identification code printed by the control of the medical staff interface 3. As an example, the subject person identification code 1030 may also be an identification code printed by the control of the medical staff interface 3, and then attached to a questionnaire of the subject person.

Although not shown, for the first waiting step S110, basic management procedures such as booting of the vaccine management device 1, network access, firmware over the air (FOTA), and/or enhanced machine-type communication (eMTC) reset may be performed in advance or periodically.

In addition, in the first waiting step S110, in addition to the subject person identification code 1030, a medical staff identification code may also be recognized. The medical staff identification code may be an identification code of a vaccine prescriber (e.g., a doctor), an identification code of doctor or nurse who extracts the vaccine and/or inoculates the vaccine, etc., but is not limited thereto. When a new medical staff identification code is recognized, the subject person identification code 1030 and the vaccine identification code 2030 recognized later may be grouped and managed with the newly recognized medical though the new medical staff identification code. Even identification code is recognized, the vaccine management device 1 may still operate in the first waiting step S110. That is, converting from the first waiting step S110 to the second waiting step S130 may be performed only when the subject person identification code 1030 is recognized.

In the first determination step S120, it may be determined whether the first identification code recognized in the first waiting step S110 is a code for identifying a subject person to be inoculated with the vaccine, i.e., the subject person identification code 1030. That is, in the first determination step S220, it may be determined whether a format of the recognized first identification code is a format of the subject person identification code.

Specifically, when the recognized first identification code is the subject person identification code 1030, the vaccine management device 1 may operate in the second waiting step S130 of waiting the recognition of the vaccine identification code 2030 in order to identify the vaccine to be inoculated into the subject person.

Meanwhile, when the recognized first identification code is not the subject person identification code 1030 unlike the above description (as an example, when the first identification code is the vaccine identification code 2030 or other wrong identification codes), the vaccine management device 1 may notify, to the medical staff, that the first identification code is not the subject person identification code 1030 through a notification means (for example, a state notification display 140 and/or a state notification speaker (not shown) of FIG. 5) provided in the vaccine management device 1. Further, when the first identification code is not the subject person identification code 1030, the vaccine management device 1 may operate in the first waiting step S110 without step converting to the second waiting step S130.

The second waiting step S130 (or a second waiting mode) may be a step in which the identification code recognition unit of the vaccine management device 1 waits recognition of the vaccine identification code 2030. That is, when the second identification code is recognized in the second waiting step S130, it may be determined in the second determination step (S140) whether the second identification code is a code for identifying a vaccine product accommodated in a vaccine syringe, i.e., the vaccine identification code 2030. That is, it may be determined in the second determination step S140 whether a format of the recognized second identification code is a format of the vaccine identification code.

Meanwhile, vaccine products identified in the second waiting step S130 and the second determination step S140 may be diversified for each vaccine inoculation system and/or for each medical site. As an example, the identified vaccine products may be a prefilled syringe as shown in FIG. 5, and in this case, the medical staff may identify the prefilled syringe, and then inoculate the vaccine into the subject person. As another example, the identified vaccine product may be a vial unlike that shown in FIG. 5, and in this case, the medical staff may identify the vial, and extract the vaccine from the vial by using the syringe, and then inoculate the vaccine into the subject person. As still another example, the identified vaccine product may be a syringe into which the vaccine is extracted from the vial and filled, and in this case, the medical staff may identify the syringe in which the vaccine is extracted and filled, and then inoculate the vaccine into the subject person. In this case, the vaccine identification code may be printed and attached to the syringe in which the vaccine is extracted and filled, and the vaccine identification code may also include unique information (e.g., a product name, a distribution history, a shelf life, etc.) of the vial, and prescription/inoculation information (e.g., prescription/inoculation-related place information, medical staff information, prescription amount information, inoculation amount information, etc.).

The vaccine management device 1 may maintain the second waiting mode S130 of waiting the recognition of the vaccine identification code 2030 only for a specific time. For example, when it is determined in the first determination step S120 that the first identification code is the subject person identification code 1030, the vaccine management device 1 may operate a first counter that counts a first specific time (e.g., 30 seconds), and maintain the second waiting step S130 only for the first specific time.

When the second identification code recognized during the second waiting step S130 is determined to be the vaccine identification code 2030, a merged information transmission step S150 to be described below may be performed, and when the second identification code 2030 is not recognized during the second waiting step S130, the vaccine management device 1 may discard the subject person identification code (i.e., information included in the subject person identification code), and then return to the first waiting step S110. Only when the first identification code is the subject person identification code 1030 and the second identification code is the vaccine identification code 2030, the vaccine management device 1 may transmit the merged data to the management server 2.

Meanwhile, according to the embodiment, when the second identification code recognized in the second waiting step S130 is not the vaccine identification code 2030 but the subject person identification code 1030, the first counter may be reset. That is, an operating time of the second waiting step S130 may be extended.

As a specific example, it is assumed that in the first waiting step S110, a subject person identification code of subject person X is recognized and in the second waiting step S130, a subject person identification code of subject person Y is recognized. In this case, when the operating time of the second waiting step S130 is updated, and a vaccine identification code of vaccine A is recognized in the updated second waiting step S130, the vaccine management device 1 discards an identification code of subject person X, and the subject person identification information of subject person Y and the vaccine identification information of vaccine A are merged and transmitted to data merged and transmitted in the merged information transmission step S150.

In the merged information transmission step S150, the vaccine management device 1 may merge the subject person identification information corresponding to the subject person identification code 1030 and the vaccine identification information corresponding to the vaccine identification code 2030, and transmit merged data to the management server 2. As shown, the merged information transmissions step S150 may be performed only when the subject person identification code 1030 is normally recognized in the first waiting step S110, and the vaccine identification code 2030 is normally recognized in the second waiting step S130. That is, the vaccine management device 1 does not transmit data to the management server 2 until the identification information of a specific subject person and the identification information of a specific vaccine are collected.

Meanwhile, the merged data transmitted in the merged information transmission step S150 may include additional information in addition to the subject person identification information and the vaccine identification information. The subject person identification information, for example, the additional information may be medical identification information corresponding to the medical staff identification code, various biometric information such as a blood temperature, a blood pressure, a heart rate, etc., of the subject person, which are measured in medical sites, etc. The vaccine identification information may include product information and manufacturing information (e.g., manufacturer, lot number, manufacturing number, manufacturing date, etc.) of vaccines.

Capacities of the subject person identification information and the vaccine identification information included in the merged data may be 28 bytes to 70 bytes. In addition, the subject person identification information may be subject person information de-identification data. When the capacities of the subject person identification information and the vaccine identification information are within the numerical number r range, personal information of the subject person is de-identified as described above, thereby increasing personal information security, and minimizing constraints according to a transmission capacity and a transmission speed in a poor wired/wireless communication environment.

The merged data transmitted in the merged information transmission step S150 may be transmitted after encoding for encryption, and in this case, the management server 2 may decode and manage received data.

For example, according to the present invention, an operating algorithm of the vaccine management device 1 may be simplified, and further, unnecessary data transmission/reception between the vaccine management device 1 and the management server 2 may be minimized, and optimal data management may be performed by a minimum data amount.

Meanwhile, ‘data transmission from the vaccine management device 1 to the management server 2’ in this specification is not limited only to performing data communication to the management server 2 directly by the vaccine management device 1. That is, the vaccine management device 1 may directly communicate with the management server 2, but may also communicate with the management server 2 via a communication link such as a share device, a relay, an access point, a gateway, a router, and/or a sub-management server.

Specifically, a plurality of vaccine management devices 1 grouped according to a predetermined criterion, for example, a plurality of vaccine management devices 1 provided in the same medical sites may perform communication with a communication link separately provided in relevant medical sites, and communication links provided in each of the plurality of medical sites may perform communication with the management server 2.

As an example, wired communication may be performed between the plurality of vaccine management devices 1 and the communication link, and the wired communication may also be performed between the communication links and the management server 2. As another example, short-range wireless communication may be performed between the plurality of vaccine management devices 1 and the communication link, and the wired communication may be performed between the communication links and the management server 2. As still another example, the short-range wireless communication may be performed between the plurality of vaccine management devices 1 and the communication link, and mobile communication may be performed between the communication links and the management server 2. As yet another example, the wired communication may be performed between the plurality of vaccine management devices 1 and the communication link, and the mobile communication may be performed between the communication links and the management server 2.

FIG. 4 is a diagram exemplarily illustrating a method for generating a subject person identification code used in the vaccine management system according to some embodiments of the present invention.

Referring to FIG. 4, the input device 1000 may directly/indirectly receive the identity information input from the subject person, and transmit the input information to the management server 2. In some embodiments, the identity information may include a name, a gender, and a contact point of the subject person. Here, the input device 1000 may be the subject person terminal or the medical staff terminal, and when the input device 1000 is the subject person terminal, the subject person interface 4 may directly receive the identity information input from the subject person, and when the input device 1000 is the medical staff terminal, the subject person interface 4 may also indirectly receive the identity information input confirmed from the subject person through the medical staff interface 3.

The management server 2 may convert the received subject person identity information into the subject person identification code 1030, and transmit the converted subject person identification code 1030 to the output device 2000. Here, the management server 20 may encrypt or de-identify and convert the received identity information into the subject person identification code 1030. That is, the management server 2 may exclude and then transmit personal information of the subject person included in the identity information of the subject person to the output device 2000, and the de-identified subject person identification code may also be re-identified in the medical staff interface 3 and/or the vaccine informatization system (e.g., health insurance management system, DUR, etc.) if necessary in the future.

The output device 2000 may output the received subject person identification code 1030, and the output subject person identification code 1030 may be thereafter identified through the vaccine management device 1. According to the embodiment, the output device 2000 may be the printing device such as the identification code printer provided in medical sites or the subject person terminal display implementing the subject person interface 4. That is, the subject person identification code 1030 may be output in printout in medical sites or may be output by the subject person terminal display.

Meanwhile, in the case of some vaccines (in particular, Corona 19 vaccine), two or more vaccine inoculations are required, and when a specific vaccine is inoculated, and then other kinds of vaccines are inoculated interchangeably, there are problems such as no effect of vaccine inoculation or occurrence of side effects. Therefore, the vaccine management device 1 and a system operating method of the present invention to prevent interchangeable inoculation will be described.

FIG. 7 is a flowchart for describing an operating method of a vaccine management device 1 according to another embodiment of the present invention. FIG. 7 exemplarily illustrated a method for managing whether the subject person is previously inoculated with the vaccine, which is performed in the management server 2 and whether the previously inoculated vaccine and vaccine to be inoculated later (i.e., vaccine identified in the vaccine management device 1) are compatible with each other.

Referring to FIGS. 5 and 7, when the vaccine management device 1 transmits the data obtained by merging the subject person identification information and the vaccine identification information (S150), the management server 2 may determine whether the identified vaccine is suitable for vaccine inoculation based on the received merged data.

Specifically, in the merged information transmission step S150, the vaccine management device 1 may transmit the data obtained by merging the subject person identification information and the vaccine identification information, and then operate a second counter counting a second specific time (e.g., 5 seconds). Until the second counter is terminated after the merged information transmission step S150, a suitability waiting step S210 of waiting reception of vaccine inoculation suitability information from the management server 2 may be performed. The vaccine inoculation suitability information may be information indicating whether it is appropriate to inoculate a specific vaccine corresponding to the vaccine identification information into a specific subject person corresponding to the subject person identification information.

When there is no response from the management server 2 during the suitability waiting step S210, the vaccine management device 1 may output a second notification for notifying a communication error with the management server 2 (S212), and return to the second waiting mode S130 again and operate for the first specific time.

When vaccine inoculation suitability information from the management server 2 is received during the suitability waiting step S210, the vaccine management device 1 may output the notification for the vaccine inoculation suitability information. That is, when information for notifying that vaccine inoculation is suitable is received, the vaccine management device 1 may output a third notification for notifying the information (S213), and when information for notifying that the vaccine inoculation is unsuitable is received, the vaccine management device 1 may output a fourth notification for notifying the information (S214). The medical staff may confirm again whether vaccine to be inoculated is suitable through the vaccine management device 1 based on the third and fourth notifications.

To this end, the management server 2 may determine the received vaccine inoculation suitability based on the subject person identification information received from the vaccine management device 1, an inoculation status (whether the vaccine is previously inoculated and the type of previously inoculated vaccine) of the subject person stored in the management server 2, and distribution and storage history information of individual vaccines stored in the management server 2. Here, the vaccine inoculation suitability may mean a vaccine inoculation compatibility between the previously inoculated vaccine and a vaccine based on newly received information (i.e., a vaccine to be inoculated), or the coldchain history suitability of individual vaccines. For example, when a vaccine previously inoculated into a specific subject person is A company's vaccine, a newly received vaccine is B company's vaccine, and there is no compatibility between A company's vaccine and B company's vaccine, the management server 2 may determine that it is not suitable to inoculate B company's vaccine into the specific subject person.

FIG. 8 is a flowchart for describing an operating method of a vaccine management device 1 according to still another embodiment of the present invention. FIG. 8 exemplarily illustrates a method for directly determining, by the vaccine management device 1, whether the subject person is previously inoculated with the vaccine and whether the previously inoculated vaccine and vaccine to be inoculated (i.e., vaccine identified in the vaccine management device 1) are compatible with each other.

Referring to FIGS. 5 and 8, when the subject person identification code 1030 is recognized in the first waiting step S110 (S120), a previous vaccine inoculation history of the subject person may be obtained based on the recognized subject person identification code 1030 (S310). To this end, the subject person identification code 1030 may additionally include inoculation information-related data of the subject person. For example, the subject person identification code 1030 may additionally include specific fields in which when the subject person is an initial inoculator, it refers to “0”, when the previously inoculated vaccine is A company's vaccine, it refers to “01”, when the previously inoculated vaccine is B company's vaccine, it refers to “10”, and when the previously inoculated vaccine is C company's vaccine, it refers to “11”, respectively.

Based on the obtained previously inoculated vaccine history, when the subject person is initially inoculated with the vaccine, the vaccine management device 1 operates in the second waiting mode S130, and when the subject person is inoculated with the vaccine one time or more, the vaccine management device 1 may operate in a third waiting mode S320 preceding to perform the vaccine inoculation suitability determination.

Here, the third waiting mode S320 may be performed in substantially the same manner as the second waiting mode S130, but as shown in FIG. 8, a step S340 of determining the vaccine inoculation suitability before transmitting data to the management server 2 after the vaccine identification code 2030 is scanned may be additionally performed.

That is, when the vaccine identification code 2030 is recognized in the third waiting mode S320 for a specific time (e.g., 30 seconds), the vaccine inoculation suitability determination step S340 may be performed, and when the vaccine identification code 2030 is not recognized, the vaccine management device 1 may discard the subject person identification information obtained in the first determination step S120 and return to the first waiting step S110.

In the vaccine inoculation suitability determination step S340, a process of determining whether it is suitable to inoculate a specific vaccine into a specific subject person may be performed based on the previous vaccine inoculation history of the specific subject person, and the merged information transmission step S150 is performed only when a determination result in the vaccine inoculation suitability determination step S340 is suitable. That is, when the vaccine inoculation is determined to be suitable, the vaccine management device 1 outputs the third notification for notifying the determination (S343), and then merges and transmits the specific subject person and the specific vaccine information to the management server 2, and when the vaccine inoculation is determined to be unsuitable, the vaccine management device 1 outputs a fourth notification for notifying the determination (S344), and does not transmit data to the management server 2.

Through the above operation, only the inoculation history information is added to the subject person identification code, thereby minimizing the number and the amount of communications for each communication between the vaccine management device 1 and the management server 2, and at the same time, rechecking whether the vaccine to be inoculated is suitable through the vaccine management device 1.

Referring back to FIG. 2, the management server 2 may generate the vaccine inoculation history based on the subject person identification information (subject person information de-identification data) and the vaccine identification information (vaccine information data) included in the merged data transmitted from the vaccine management device 1 S50, and transmit a vaccine inoculation history message to a subject person terminal based on the generated vaccine inoculation history S60. The management server 2 may identify the basic information of the subject person to be inoculated from the subject person information de-identification data. The vaccine inoculation history may include various information such as identity information (i.e., personal information or false name information) of the subject person (i.e., an inoculated person), an inoculation date, an inoculation place, an inoculated vaccine name, an inoculation type, manufacturing information (e.g., manufacturer, lot number, manufacturing number, manufacturing date, etc.) of the inoculated vaccine.

The management server 2 may perform post management of the subject person through self-diagnosis management periodically when a predetermined time elapsed after vaccine inoculation. In step S70 of self-diagnosis managing the subject person, the management server 2 may transmit, to the subject person terminal, the self-diagnosis questionnaire for the vaccine inoculation at a second time point (e.g., 12:00) at which a predetermined first time (e.g., 3 hours) elapsed from a first time point (e.g., 09:00) of transmitting the vaccine inoculation history message. The subject person may create the received self-diagnosis questionnaire and reply to the management server 2, and the management server 2 may calculate a self-diagnosis result based on the self-diagnosis questionnaire created by the subject person, and transmit the self-diagnosis result to the subject person terminal.

Meanwhile, step S70 of self-diagnosis managing the subject person may be performed multiple times. For example, the management server 2 may transmit, to the subject person terminal, a first self-diagnosis questionnaire at the second time point at which the predetermined first time elapsed from the first time point of transmitting the vaccine inoculation history message, transmit, to the subject person terminal, a second self-diagnosis questionnaire at a third time point at which a predetermined second time elapsed from the second time point, and transmit, to the subject person terminal, and a self-diagnosis notice for notifying that there is no side effect of vaccine inoculation at a fourth time point (e.g., a time point reaching an antibody formation stabilization period) at which a predetermined fourth time elapsed from the third time point.

According to the embodiment, the management server 2 may collect the vaccine inoculation history and the self-diagnosis result of the subject person, and calculate statistics S80. That is, the vaccine healthcare solution according to an embodiment of the present invention may provide, to a vaccine inoculation provider, statistical analysis data for a real-time vaccine demand status and the side effect through step S80. Accordingly, an inoculation management institution may check a vaccine supply amount, a vaccine exhaust amount, a vaccine inventory amount (a subtotal for each management institution/for each region/for each vaccine product, and a total vaccine inventory amount), a reserve vaccine amount, emergency inoculation of vaccine, counting information of side effect vaccine, back tracking of the side effect vaccine, etc. That is, it is possible to implement a centralized vaccine control system.

In particular, the counting information of the side effect vaccine may include the subject person's side effect reaction time after inoculation for each gender, for each age, and for each product obtained through self-diagnosis, the type of side effect, etc. The counting information of the side effect vaccine may be provided as statistical numerical values to the inoculation management institution. Data on occurrence status of side effects such as side effect vaccine back tracking and recovery may also be counted.

Referring to FIG. 9, the management server 2 may provide various services for each demander of vaccine related information (e.g., inoculation subject person, medical staff, disease management institution, etc.) based on data collected from the vaccine management device 1 (subject person's basic information, vaccine product information, vaccine manufacturing information, self-diagnosis details, subject person's biometric information, subject person's entry and exit history, medical staff information, etc.) and/or the medical staff interface 3.

As described above, the management server 2 may generate and manage the subject person identification code 1030 based on the subject person's identity information, and merge and manage the vaccine identification information and the subject person identification information, and further process collected data and provide a demander-wise customized solution. As an example, the management server 2 may provide, to the medical staff interface 3, information such as a total amount, an exhaust amount, an inventory amount, coldchain history suitability, etc., of vaccine input for each medical site, or provide an automatic ordering management service of vaccines based on the information. As another example, the management server 2 may transmit, to the subject person interface 4, a vaccine inoculation history such as a hospital name, a vaccine inoculation name, a syringe product name, etc., an entry and exit history, a body temperature history, the self-diagnosis questionnaire, future vaccine inoculation date notification, etc., and provide, to the disease management institution 5, a vaccine quantity and statistical information related thereto that are received and collected from the plurality of vaccine management devices 1 and the plurality of medical sites.

FIG. 10 is a diagram exemplarily illustrating a method for managing the subject person identification code 1030 used in the vaccine management system according to an embodiment of the present invention.

Referring to FIG. 10, the subject person interface 4 may receive personal information input capable of confirming an identity from the subject person, a contact point, etc., and transmit the received information to the management server 2. The medical staff may collect, manage, and transmit the entry and exit history of the subject person (e.g., the subject person's body temperature measured at the time of visiting a hospital for inoculation, medical staff information, etc.) and a previous treatment record (in the case of a previously inoculated person) to the management server 2.

The management server 2 may generate or update the subject person identification code 1030 based on the subject person's input identity and related information. As an example, the management server 2 may perform each of new issuance of a subject person identification code for the subject person having no issuance history of the subject person identification code 1030, and update of the subject person identification code for the subject person having the existing issuance history.

The management server 2 may transmit, to the medical staff interface 3 and/or the subject person interface 4, the issued or updated subject person identification code.

The subject person interface 4 may display the received subject person identification code 1030 through the subject person terminal display. Alternatively, the subject person identification code 2030 received by the medical staff interface 3 may be printed and used for subject person identification of the vaccine management device 1.

The displayed or printed subject person identification code may be used for the subject person identification code of the vaccine management device 1 and subject person identification in subject person entry/exit history management in the hospital.

Meanwhile, additional information other than the above-described information may be transmitted and received through the information communication network 10.

For example, the management server 2 may communicate only minimum information (the subject person identification information, the vaccine identification information, and inoculation history) described above with the vaccine management device 1, and perform communication for other subject person's inoculation-related information (body temperature during hospital visit, previous vaccine inoculation details, self-diagnosis details collection, future vaccine inoculation period information, etc.) and/or coldchain logistics history information with the medical staff interface 3 and/or the subject person interface 4. Through this, limitations such as hardware constraints, manufacturing unit price, etc. of the vaccine management device 1 can be easily overcome, and stabilities of vaccine inoculation and vaccine management can be further maximized.

Further, the management server 2 additionally determines coldchain history suitability of vaccines identified by the vaccine identification code 2030, and when the vaccine management device 1 transmits the vaccine identification information to the management server 2, the management server 2 may transmit data on distribution history suitability to the vaccine management device 1.

Specifically, the management server 2 may track and manage main matters such as temperature and humidity for each interval during a distribution process of vaccines in connection with the vaccine identification code 2030, and when determining that the distribution process is unsuitable, the management server 2 may transmit vaccine distribution unsuitability information to the vaccine management device 1 immediately after the merged data including the vaccine identification information from the vaccine management device 1. When the vaccine management device 1 receives the vaccine distribution unsuitability information, the vaccine management device 1 may notify that the vaccine of the scanned vaccine identification code 2030 is unsuitable for vaccines to the medical staff through the notification means, etc.

It may be understood that those skilled in the art related to the embodiment may implement the embodiment in a modified form without departing from the essential characteristics of the above descriptions. Therefore, the disclosed methods should be considered in an illustrative viewpoint rather than a restrictive viewpoint. The scope of the present invention is defined by the claims rather than the foregoing description, and all differences within the equivalent scope should be construed as being included in the present invention.

1. An operating method of a vaccine management device including an identification code recognition unit, comprising:
a first waiting step of waiting, by the identification code recognition unit, recognition of an identification code;
a first determination step of determining, when a first identification code is recognized in the first waiting step, whether the first identification code is a subject person identification code of a vaccine inoculation subject person;
a second waiting of step waiting, when the first identification code is the subject person identification code, recognition of a vaccine identification code;
a second determination step of determining, when a second identification code is recognized in the second waiting step, whether the second identification code is the vaccine identification code; and
a merged information transmission step of transmitting, when the second identification code is the vaccine identification code, data obtained by merging subject person identification information corresponding to the subject person identification code and vaccine identification information corresponding to the vaccine identification code, to a management server.

2. The operating method of a vaccine management device of claim 1, wherein,
when the first identification code is the vaccine identification code, the vaccine management device outputs a first notification for notifying that the first identification code is a wrong identification code, and operates by returning to the first waiting step.

3. The operating method of a vaccine management device of claim 2, further comprising:
operating, between the first determination step and the second waiting step, a first counter counting a first specific time when the first identification code is the subject person identification code,
wherein the second waiting step is performed only until the first counter is terminated after the first specific time elapsed, and
wherein when the vaccine identification code is not recognized until the first counter is terminated, the subject person identification information is discarded and the vaccine management device operates by returning to the first waiting step.

4. The operating method of a vaccine management device of claim 3, wherein,
in the merged information transmission step, a second counter counting a second specific time after transmitting the data obtained by merging the subject person identification information and the vaccine identification information is operated, and
after the merged information transmission step, further comprising:
a suitability waiting step of waiting reception of vaccine inoculation suitability information indicating whether it is suitable to inoculate a specific vaccine corresponding to the vaccine identification information into a specific subject person corresponding to the subject person identification information from the management server until the second counter is terminated; and
a vaccine inoculation suitability notification step of outputting a notification for the vaccine inoculation suitability information.

5. The operating method of a vaccine management device of claim 4, wherein,
in the suitability waiting step, when the vaccine inoculation suitability information is not received until the second counter is terminated, the vaccine management device operates by returning to the second waiting step.

6. The operating method of a vaccine management device of claim 3, further comprising:
an inoculation history acquisition step of acquiring a previous inoculation history of the specific subject person corresponding to the subject person identification information based on the subject person identification information, before the second waiting step after the first determination step; and
a vaccine inoculation suitability determination step of determining whether it is suitable to inoculate the specific vaccine corresponding to the vaccine identification information into the specific subject person based on the previous inoculation history of the specific subject person acquired in the inoculation history acquisition step, before the merged information transmission step after the second determination step, wherein the merged information transmission step is performed only when it is determined that it is suitable to inoculate the specific vaccine into the specific subject person in the vaccine inoculation suitability determination step.

7. A vaccine management system, comprising:
a management server;
at least one vaccine management device; and
at least one inoculation subject person terminal,
wherein the inoculation subject person terminal collects, from an inoculation subject person, basic information of the inoculation subject person,
wherein the management server generates an inoculation subject person identification code based on the basic information of the inoculation subject person, and transmits the inoculation subject person identification code to the inoculation subject person terminal,
wherein the vaccine management device sequentially scans the inoculation subject person identification code output to the inoculation subject person terminal and a vaccine identification code provided in a vaccine, and transmits, to the management server, merged data including inoculation subject person information de-identification data acquired from the inoculation subject person identification code and vaccine information data acquired from the vaccine identification code, and
wherein the management server generates a vaccine inoculation history of the inoculation subject person based on the inoculation subject person information de-identification data and the vaccine information data, and transmits, to the inoculation subject person terminal, a vaccine inoculation history message including the vaccine inoculation history.

8. A vaccine healthcare solution providing method, comprising:
collecting, by an inoculation subject person terminal, from an inoculation subject person, basic information of the inoculation subject person;
generating, by a management server, an inoculation subject person identification code based on the basic information of the inoculation subject person;
transmitting, by the management server, the inoculation subject person identification code to the inoculation subject person terminal;
sequentially scanning, by a vaccine management device, the inoculation subject person identification code output to the inoculation subject person terminal and a vaccine identification code provided in a vaccine;
transmitting, by the vaccine management device, to the management server, merged data including inoculation subject person information de-identification data acquired from the inoculation subject person identification code and vaccine information data acquired from the vaccine identification code;
generating, by the management server, a vaccine inoculation history of the inoculation subject person based on the inoculation subject person information de-identification data and the vaccine information data; and
transmitting, by the management server, to the inoculation subject person terminal, a vaccine inoculation history message including the vaccine inoculation history.

9. The vaccine healthcare solution providing method of claim 8, further comprising:
transmitting, by the management server, to the inoculation subject person terminal, a vaccine inoculation self-diagnosis questionnaire at a second time point at which a predetermined first time elapsed from a first time point of transmitting the vaccine inoculation history message;
transmitting, by inoculation subject person terminal, to the management server, the vaccine inoculation self-diagnosis questionnaire created by the inoculation subject person;
calculating, by the management server, a self-diagnosis result based on the created vaccine inoculation self-diagnosis questionnaire; and
transmitting, by the management server, the calculated self-diagnosis result to the inoculation subject person terminal.

10. The vaccine healthcare solution providing method of claim 9, wherein the collecting the basic information of the inoculation subject person includes:
scanning, by the inoculation subject person terminal, a uniform resource locator (URL) identification code for accessing a basic information collection interface, and
collecting, by the basic information collection interface, the basic information of the inoculation subject person from the inoculation subject person.

11. The vaccine healthcare solution providing method of claim 10, wherein,
in the generating the inoculation subject person identification code, the management server de-identifies the basic information of the inoculation subject person, and
in the generating the vaccine inoculation history, the management server identifies the basic information of the inoculation subject person from the inoculation subject person information de-identification data transmitted from the vaccine management device.

12. The vaccine healthcare solution providing method of claim 11, wherein,
in the transmitting the merged data to the management server, capacities of the inoculation subject person information de-identification data and the vaccine information data included in the merged data are 28 to 70 bytes.

13. The vaccine healthcare solution providing method of claim 12, wherein,
in sequentially scanning the inoculation subject person identification code and the vaccine identification code, the vaccine management device operates to include:
a first waiting step of waiting, by an identification code recognition unit provided in the vaccine management device, recognition of an identification code,
a first determination step of determining, when a first identification code is recognized in the first waiting step, whether the first identification code is the inoculation subject person identification code,
a second waiting step of waiting, when the first identification code is the inoculation subject person identification code, recognition of the vaccine identification code, and
a second determination step of determining, when a second identification code is recognized in the second waiting step, whether the second identification code is the vaccine identification code, and
in the transmitting the merged data to the management server, the vaccine management device transmits the merged data to the management server only when the first identification code is the inoculation subject person identification code and the second identification code is the vaccine identification code.
